(19) United States
(12) Patent Application Publication
(10) Pub. No.: US 2008/0139951 A1
(43) Pub. Date: Jun. 12, 2008

(54) Detection of Stenosis

(75) Inventors: Abhilash Patangay, Inver Grove Heights, MN (US); Marina Brockway, Shoreview, MN (US); Jeffrey E. Stahmann, Ramsey, MN (US); Yi Zhang, Blaine, MN (US); Gerrard M. Carlson, Champlin, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/608,578

(22) Filed: Dec. 8, 2006

(51) Int. Cl. A61B 5/026; A61F 2/82

(52) U.S. Cl. 600/504; 623/1.15

(57) ABSTRACT

A system for detecting stenosis in a patient. The system includes an implantable sensing unit having a turbulence sensor and a communication device for transmitting a signal from the turbulence sensor. The system also includes a cardiac sensor for generating a signal corresponding to cardiac activity and a processing device configured to receive signals from the sensing unit and from the cardiac sensor. The processing device is configured to determine a time window corresponding to cardiac activity, to determine a turbulence level from the turbulence signal within the time window, and to detect the presence of stenosis from the turbulence level.

### FIELD OF THE INVENTION

The invention relates to the detection of stenosis within a blood vessel, and more particularly, to implantable and intracorporeal devices for detecting stenosis within a blood vessel.

### BACKGROUND OF THE INVENTION

Stenosis of blood vessels is a major health concern. Stenosis is the partial or nearly complete blocking of a blood vessel, also called an occlusion of a blood vessel. Stenosis typically results from the build-up of plaque and cholesterol within a blood vessel. Although stenosis can occur in any of the blood vessels within a person's body, a particular concern is stenosis within the coronary and carotid blood vessels. For example, a stenosis of a coronary artery can result in a reduction of the blood flow to the heart muscle, possibly resulting in angina or a heart attack. Because the consequences of an occluded blood vessel are severe and include the possibility of death, and because therapy exists to treat an occluded blood vessel, such as a stent or a bypass surgery, it is often desirable to be able to detect stenosis in a patient.

Various methods exist for detecting stenosis. One way of detecting stenosis is an angiogram. An angiogram requires inserting a catheter into a blood vessel and releasing a radiocontrast agent (such as iodine) into the bloodstream. In the presence of the radiocontrast agent, the blood vessel is viewed with an x-ray machine. The radiocontrast agent within the blood allows the inner surface of the blood vessel to be visible on the x-ray image. Although this procedure allows accurate determination of whether stenosis is present, it does have certain limitations. For example, an angiogram is only capable of indicating the status of blockage or stenosis at the single point in time when the procedure is performed. However, a patient's condition may change over time, and it is desirable to be able to detect a change in the patient's condition in order to provide a therapy in a timely fashion, as well as to be able to monitor the efficacy of any administered therapy. Angiograms also have medical risks and drawbacks, including possible allergic reactions to the contrast material, possible tissue damage from the catheter, and the exposure to x-ray radiation.

Another method for detecting stenosis relies on a microphone, accelerometer, or other transducer that is positioned on the patient's skin to sense cardiac sounds. It is generally known that blood flowing through an occluded or partially occluded vessel tends to transition from laminar flow to turbulent flow as it travels into, through, and past a restriction. It is also known that turbulent blood flow tends to generate an acoustic wave that propagates through the patient's body tissue and can be sensed at the patient's skin. These acoustic waves have very low sound pressure levels (on the order of −100 dB) and also occur across an extended frequency range that includes moderately high frequencies (up to about 1.2 kHz). These acoustic waves tend to be attenuated by the body tissue, particularly at higher frequencies, and therefore require transducers having very high sensitivity to measure. However, such transducers do exist and can be used successfully to detect stenosis. For example, see Padmanabhan et al., Accelerometer Type Cardiac Transducer for Detection of Low Level Heart Sounds, IEEE Transactions on Biomedical Engineering, Vol. 40, No. 1, January 1993. However, there are limitations associated with the technique of measuring heart sounds at the patient's skin. For one, the accuracy of detection can be affected by the presence of ambient sounds, and therefore the technique must occur in a very quiet room, often times a room that has special acoustical properties. Although the procedure for taking measurements is less invasive than an angiogram, and therefore more readily conducted on a patient, the technique is not adapted to continuous monitoring of a patient's condition and therefore is not well-suited for detecting changes in stenosis. The signal to noise ratio can also be low, in part because of the attenuation of sound waves within the body tissues, which can result in lower diagnostic accuracy.

Improved techniques for detecting stenosis are needed.

### SUMMARY OF THE INVENTION

One aspect of the invention relates to a system for detecting stenosis in a patient. The system includes an implantable sensing unit that has a turbulence sensor and also has a communication device for transmitting a signal from the turbulence sensor. The system also includes a cardiac sensor for generating a signal corresponding to cardiac activity and a processing device that is configured to receive signals from the sensing unit and from the cardiac sensor. The processing device is configured to determine a time window corresponding to cardiac activity, to determine a turbulence level from the turbulence signal within the time window, and to detect the presence of stenosis from the turbulence level.

Another aspect of the invention relates to a method of detecting stenosis in a patient. The method includes the steps of sensing turbulence that occurs within a blood vessel using an implanted sensing unit, sensing the patient's cardiac activity, transmitting signals representing turbulence and cardiac activity to a processing device, and analyzing the turbulence and cardiac activity signals within the processing device to determine a time window corresponding to cardiac activity, determining a turbulence level from the turbulence signal within the time window, and detecting the presence of stenosis within a blood vessel.

Yet another aspect of the invention relates to a stent system. The stent system includes an expandable, generally cylindrical structure configured to be placed in a body lumen and to exert radial pressure on the body lumen. The stent system also includes a turbulence sensor attached to the cylindrical structure, the turbulence sensor being configured to transmit a signal, and a processing device configured to receive the signal from the turbulence senor and to analyze the signal to detect the presence of stenosis within the body lumen.

The invention may be more completely understood by considering the detailed description of various embodiments of the invention that follows in connection with the accompanying drawings.

### BRIEF DESCRIPTION OF THE DRAWINGS

FIG. 1 is a plot of the power spectrum of sound pressure waves associated with turbulent flow in a blood vessel.

FIG. 2 is a plot of the power spectrum of sound pressure waves associated with laminar flow in a blood vessel.

FIG. 3 is a schematic of an implantable medical system for detecting stenosis that is constructed according to the principles of the present disclosure.

FIG. 4 is a flow chart depicting steps of a method for detecting stenosis in a patient.

FIG. 5 is a schematic depiction of a stent system having a turbulence sensor.

FIG. 6 is a schematic of an alternative embodiment of the implantable medical system of FIG. 3.

FIG. 7 is a schematic of another alternative embodiment of the implantable medical system of FIG. 3.

FIG. 8 is a flow chart depicting steps of one example of a method of differentiating between left-sided and right-sided murmurs.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives following within the scope and spirit of the invention as defined by the claims.

### DETAILED DESCRIPTION OF THE INVENTION

Blood flowing through a vessel can be characterized as being either laminar flow or turbulent flow, or in a transition state between laminar and turbulent flows. Turbulence is flow dominated by recirculation, eddies, and apparent randomness and chaos, whereas laminar flow is characterized by flow in smooth sheets or layers. Whether a flow is laminar or turbulent is determined by the ratio of inertia forces to viscous forces within the fluid. For a fluid flowing in a conduit such as a blood vessel, the Reynold's number can be calculated to determine which flow regime is likely present. The Reynold's number is a non-dimensional quantity that is defined as:

$$R = \frac{ρ \cdot v \cdot D}{μ}$$

where ρ is the density of the fluid, ν is the velocity of the fluid, D is the diameter of the conduit, and μ is the viscosity of the fluid. Under ideal conditions where the fluid conduit is long and straight, a Reynold's number below about 2000 indicates that the fluid is laminar. From Reynold's numbers of about 2000 to 4000 the fluid is defined as being in transition between laminar and turbulent flow. A flow is considered turbulent at Reynold's numbers above about 4000. However, under non-ideal conditions, such as in the vasculature of a patient's body, where there are short distances between obstructions and turns, turbulent flow can occur at significantly lower Reynold's numbers.

When blood flows through a partially occluded blood vessel, its velocity must increase as it passes through the occlusion to maintain a given mass flow rate. If this velocity increase is great enough, the blood flow will transition from a laminar regime to a turbulent flow regime. It is important to note that the velocity of a fluid in a tube will be inversely proportional to the square of the radius of the tube. Therefore, if a blood vessel is reduced to half of its original diameter, such as through the buildup of plaque, the flow velocity will actually increase four-fold, tending to make the transition to turbulent flow more likely.

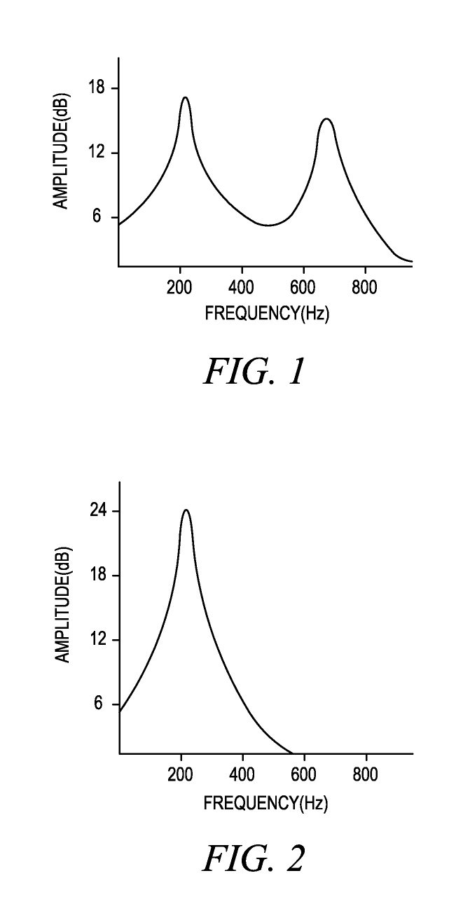

A fluid in turbulent flow tends to generate acoustic pressure waves that are different in character from the acoustic pressure waves generated by laminar flow. Turbulent flow tends to result in different pressure wave frequencies and higher sound pressure levels. For example, FIG. 1 shows an example of the power spectrum of an acoustic pressure wave of blood flow through an occluded blood vessel, where significant turbulence exists. FIG. 2 shows an example of the power spectrum of an acoustic pressure wave of blood flow through a blood vessel without occlusion, where the flow is generally laminar. Comparing FIGS. 1 and 2, it can be seen that there is a significant difference in the acoustic pressure wave signatures between turbulent flow and laminar flow, particularly in the frequencies of 200 to 800 Hz. This difference in characteristic pressure waves can be utilized to detect the presence of turbulence, which in turn provides an indication of the presence of stenosis. The signals illustrated in FIGS. 1 and 2 can be derived from a variety of sensors, including a microphone, an accelerometer, or other devices.

In addition to detecting changes in the power spectrum of pressure waves caused by blood flow turbulence, stenosis and the resulting turbulent flow affect other physiological parameters and have other characteristics that allow detection through devices other than an acoustic sensor. For example, a pressure sensor within the vasculature may be used to detect stenosis, since pressure increases in the vasculature as a blood vessel gets more occluded. As another example, a flow transducer can be used to detect the rate of non-laminar flow in a blood vessel.

The velocity of blood flowing in blood vessels varies as a function of time as the heart beats. Blood flow is generally a pulsatile flow, as opposed to a continuous flow, and accordingly there will be time periods within a single heart cycle in which the blood flow is at a maximum and other time periods where it is at a minimum. For example, blood flow in the pulmonary artery or carotid artery will tend to be maximal at ventricular systole, where the ventricular chamber pressure is greatest and the blood is being pumped most forcefully through the vessels. However, in other vessels such as the coronary artery, maximal blood flow will occur at other times. For example, in the coronary artery the greatest flow will occur at diastole, at least in part because the higher ventricular chamber pressures during systole tend to compress the blood vessels within the cardiac tissue and increase their resistance to flow, so that maximum flow occurs when this resistance drops after systole. Due to the dependence of blood flow to cardiac activity some embodiments will utilize only blood flow information gathered during certain portions of the cardiac cycle, for example the systolic portion of the cardiac cycle. Other embodiments will utilize only blood flow information gathered during diastolic portion of the cardiac cycle. In other embodiments blood flow information may be used from both systolic and diastolic portions of the cardiac cycle. Additional embodiments use blood flow from the entire cardiac cycle. All of these embodiments may use all or portions of only one cardiac cycle or all or portions of multiple cardiac cycles.

The cardiac cycle can be sensed through a number of different mechanisms. For example, measurements can be made of electrical signals that are representative of the heart electrical function. For example, these electrical signals propagate from the heart and travel through the body tissue to the body surface, where they can be measured by an electrocardiogram (ECG). These electrical signals can also be measured within the patient's body. For example, where a cardiac rhythm management device is present, there are typically one or more leads implanted in the patient's cardiac tissue that are capable of sensing the electrical activity of the heart and transmitting a signal to electronic circuitry within the device. In another example, electrical activity of the heart is measured from electrodes that are implanted outside of the patient's heart, such as subcutaneous electrodes, as described in McCabe, et al, WO2005089643 A1, WIRELESS ECG IN IMPLANTABLE DEVICES, which is incorporated herein in its entirety. As is known to a person of skill in the art, a cardiac electrical signal includes a portion designated as a T-wave, where the T-wave provides an indication of the beginning of diastole. The cardiac electrical signal also includes a portion designated as an R-wave, where the R-wave provides an indication of the beginning of the systole. The R-wave can be used to determine the beginning of diastole by selecting a time interval following the R-wave, where this time interval is determined from regression analysis of statistical studies of patients and ideally is calculated as a function of certain relevant physical characteristics such as sex of the patient and the existence of heart disease. For example, see Arnold Weissler et al., Systolic Time Intervals in Heart Failure in Man, Circulation, Vol. XXXVII, February 1968, pp. 149-159, for a method of calculating this time interval.

Heart sounds can also be used as an indication of the cardiac cycle. A first heart sound, generally designated as S₁, corresponds to the closing of the atrioventricular (AV) valves between the atria and ventricles. This heart sound therefore corresponds to the beginning of ventricular systole. A second heart sound, generally designated as S₂, corresponds to the closing of the aortic and pulmonary valves, also called the semilunar valves. This heart sound therefore corresponds to the end of ventricular systole, when the pressure in the ventricles falls below the aortic and pulmonary artery pressures, and thus also corresponds to the beginning of diastole. These heart sounds include audible and inaudible mechanical vibrations that can be sensed, for example, with an accelerometer or a microphone. Other devices may also be used to sense heart sounds.

Another type of sensor that can be used to indicate the status of the cardiac cycle is an intracardiac pressure sensor. The pulmonary artery pressure, aortic pressure, left or right atrial pressure, or left or right ventricular pressure can be used to determine the beginning of the systole and diastole.

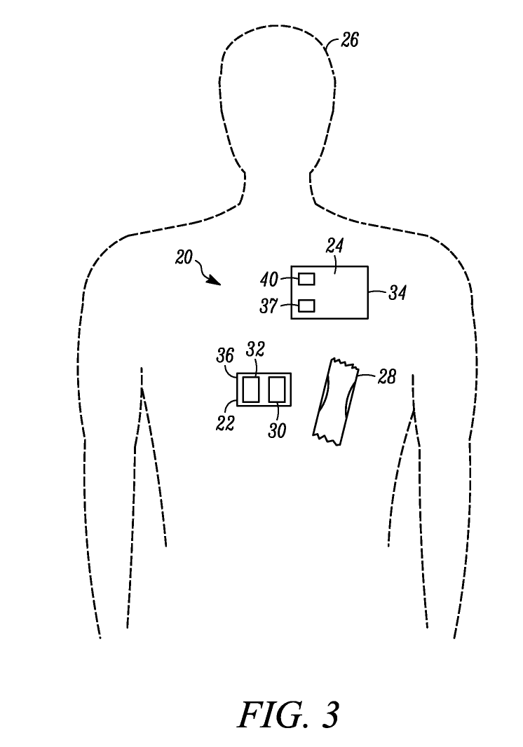

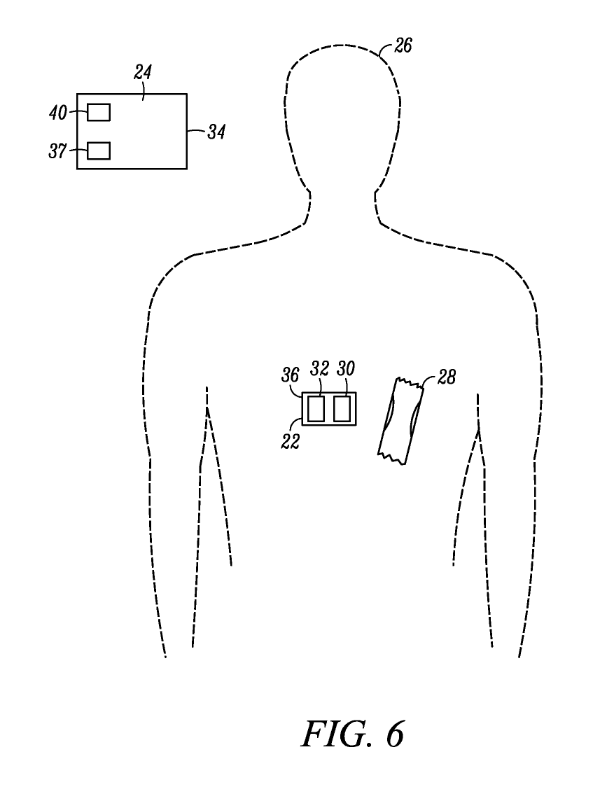

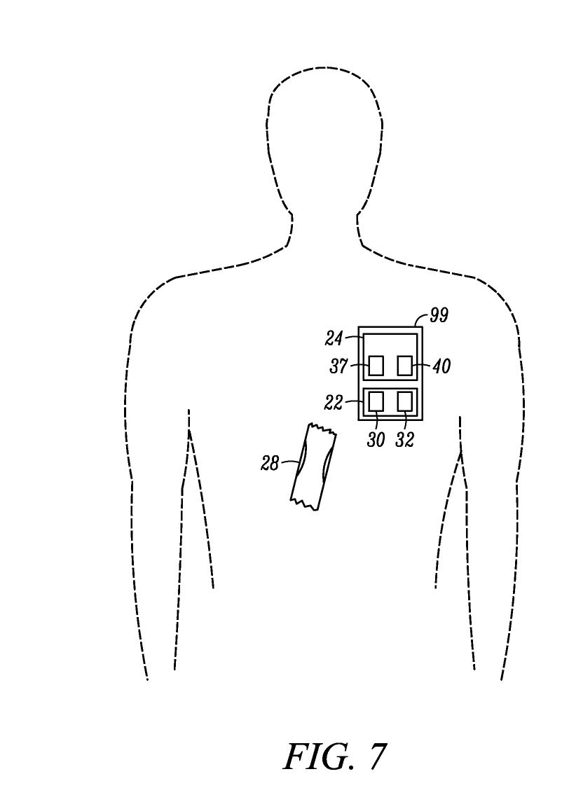

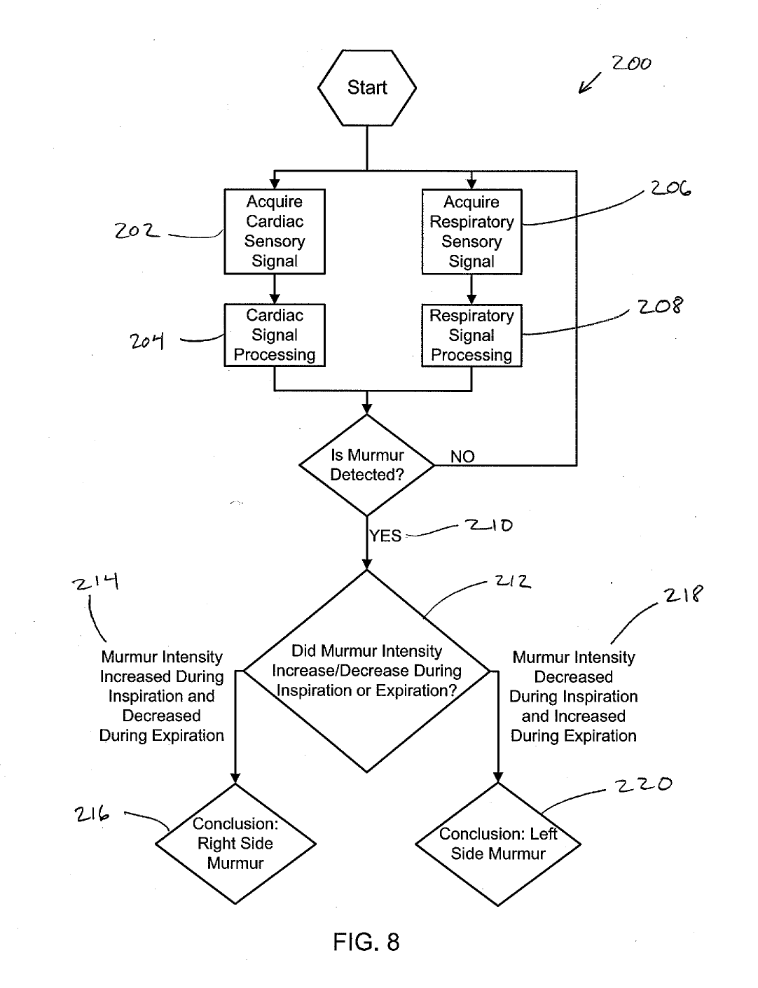

One embodiment of the invention is depicted in FIG. 3, which is a schematic drawing of an implantable stenosis detection system constructed according to the principles of the present disclosure. The stenosis detection system 20 of FIG. 3 includes an implantable sensing unit 22 and a processing device 24 that is located remotely from sensing unit 22. Sensing unit 22 includes a turbulence sensor 30 and a communication device 32. The processing device 24 includes a housing 34 which contains the subcomponents of processing device 24. Sensing unit 22 includes a housing 36 which contains turbulence sensor 30 and communication device 32. In the embodiment of FIG. 3, housing 34 of processing device 24 is separate from housing 36 of sensing unit 22. In the embodiment of FIG. 3, both sensing unit 22 and processing device 24 are shown as implanted within a patient 26. However, in other embodiments, such as the embodiment of FIG. 6, processing device 24 is not implanted within a patient 26, but instead, processing device 24 is located outside of patient 26. In another embodiment, such as the embodiment of FIG. 7, processing device 24 and implantable sensing unit 22 share a common housing 99. In one embodiment the processing device 24 is located inside an implantable device capable of delivering therapy such as a cardiac rhythm management device. Examples of cardiac rhythm devices are pacemakers, defibrillators, cardiac resynchronization devices and neural stimulation devices.

Stenosis detection system 20 further includes a cardiac sensor 37. Cardiac sensor 37 may be any of a number of sensors for monitoring the cardiac cycle. In one embodiment, cardiac sensor 37 is a microphone or accelerometer for sensing heart sounds. In another embodiment, cardiac sensor 37 is configured to sense electrical activity from the heart, such as an ECG or other analogous measurement. In one embodiment, cardiac sensor 37 is located within a processing device 24. For example, cardiac sensor 37 may include the leads of a cardiac rhythm management device that are in contact with cardiac tissue and that transmit a signal to electronic circuitry within the device. In another embodiment, cardiac sensor 37 is a stand-alone device. Cardiac sensor 37 is configured to transmit a signal to processing device 24, where the signal correlates to cardiac cycle.

A further embodiment of stenosis detection system 20 includes a respiratory sensor 40. Respiratory sensor is configured to monitor the patient's respiratory cycle. In one embodiment, respiratory sensor 40 is a microphone for sensing respiratory sounds. In another embodiment, respiratory sensor 40 is an impedance sensor for measuring changes in impedance in body tissues that correspond to the respiratory cycle. In yet another embodiment, respiratory sensor 40 is an accelerometer for sensing movement of the body due to respiration. In one embodiment, respiratory sensor 40 is located within processing device 24. In another embodiment, respiratory sensor 40 is a stand-alone device. Respiratory sensor 40 is configured to transmit a signal to processing device 24, where the signal correlates to the respiratory cycle. In yet another embodiment, communication between the processing device 24 and the respiratory sensor 40 is bi-directional.

Patient 26 has a plurality of blood vessels, and a portion of one blood vessel is depicted in FIG. 3 as vessel portion 28. Vessel portion 28 typically is selected as a portion of a blood vessel that is prone to occlusion, such as a coronary artery or a carotid artery. However, vessel portion 28 can be any portion of a blood vessel that is to be monitored for stenosis. As blood flows through vessel portion 28, pressure waves are generated by the fluid that tend to be transmitted through the surrounding body tissue, and these pressure waves tend to correspond to blood flow characteristics within the vessel. Pressure waves are defined to include any wave energy propagation from the vessel, including vibration waves, pressure waves, and acoustic waves. In some cases, vibration waves, pressure waves, and acoustic waves are synonymous and can refer to the same phenomena. In other cases these may not be synonymous, such as where an acoustic wave is present but at such a low level that no mechanical vibration is detectable. In operation, if the vessel is not significantly occluded, the blood flowing through vessel portion 28 will be laminar, which will result in turbulence sensor 30 generating a signal that can be processed to produce a characteristic pressure wave such as that shown in FIG. 2. Alternatively, if the vessel is partially occluded, the blood flowing through vessel portion 28 may be turbulent, which will result in turbulence sensor 30 generating a signal that can be processed to produce a different characteristic pressure wave such as that shown in FIG. 1.

In some embodiments, turbulence sensor 30 is located within the patient's vasculature and detects a fluid pressure at various points. In other embodiments, turbulence sensor 30 is a flow transducer (for example, as described in U.S. Pat. No. 5,873,835) that is located in the patient's vasculature, such as a hot wire anemometer-type flow transducer.

The location of sensing unit 22 depends on the type of turbulence sensor that is employed. Where the sensing unit 22 is a microphone or accelerometer, is preferably located in proximity to vessel portion 28, but not within vessel portion 28. In certain embodiments, turbulence sensor 30 of sensing unit 22 is configured to sense waves generated from vessel portion 28 that correlate to the existence of turbulence within the vessel. For example, turbulence sensor 30 may be an accelerometer that is configured to sense vibration waves that propagate from vessel portion 28. Alternatively, turbulence sensor 30 may be a microphone that is configured to sense acoustic waves that propagate from vessel portion 28. Turbulence sensor 30 may also be a pressure transducer that senses pressure within the vessel portion 28. Yet other embodiments of turbulence sensor 30 are usable. The closer that sensing unit 22 is to vessel portion 28, the less the intervening tissue will attenuate the waves and the more accurately sensing unit 22 will be able to sense the waves from vessel portion 28. However, where the sensing unit 22 is positioned further away from vessel portion 28, sensing unit 22 will have greater sensitivity to the waves from other vessels within the patient. Additionally, stenosis detection system 20 may include more than one turbulence sensor 30, where any additional turbulence sensors 30 are used to provide additional turbulence data for greater accuracy in the detection of stenosis. Communication device 32 is configured to receive a signal from turbulence sensor 30 and is further configured for transmitting a signal from sensing unit 22, where the transmitted signal is representative of the turbulence within vessel portion 28.

Processing device 24 is configured to receive signals from communication device 32 of sensing unit 22 and to receive signals from cardiac sensor 37. In one embodiment, processing device 24 is also configured to receive signals from respiratory sensor 40. Processing device 24 is configured to detect stenosis from the received signals. In one embodiment, processing device 24 is configured to determine a time window that corresponds to one complete heart beat cycle from the signal from cardiac sensor 37. In another embodiment, processing device 24 is configured to determine a time window that corresponds to the diastolic phase from the signal from cardiac sensor 37. In a further embodiment, processing device 24 is configured to correlate the measurements of turbulence and cardiac cycle to measurements of the respiratory cycle.

Processing device 24 is further configured to determine the maximum measured turbulence level within the time window, and based on the maximum measured turbulence in the time window, is configured to detect the presence of stenosis. Detecting the presence of stenosis may involve determining whether the detected turbulence exceeds a threshold for indicating the presence of stenosis, or it may involve correlating the degree of turbulence to a degree of stenosis. Detecting the presence of stenosis may also involve correlating the degree of turbulence to the respiratory cycle. For example, detecting the presence of stenosis may involve comparing the indicated turbulence level to a value or values that are obtained from patient studies that provide a correlation between indicated turbulence level and degree of stenosis. The detected presence of stenosis can be tracked in time, where the abrupt cessation of turbulence can be used to determine that there is complete occlusion. By tracking the turbulence data in time, trends can be observed that indicate stenosis. The cessation of turbulence may also indicate that stenosis has been reduced, and historical turbulence data will assist with distinguishing between the two situations. Some embodiments of the processing device include a programmer, a repeater, or both, for facilitating communication with or control of other devices. Various embodiments of processing devices discussed herein are integrated into or communicate with remote patient management systems designed to gather information from implanted devices, store the information electronically, and alert patients and/or clinicians when certain conditions are present. For example, in one embodiment of the invention, the system communicates with a remote patient management system. In one embodiment of a patient management system used in connection with a stenosis detection system, the patient and the clinician are alerted when the presence of stenosis is detected. In one embodiment, a remote patient management system provides a home monitoring device for patients that wirelessly reads implantable device information at times specified by the clinician. The data is transmitted to an Internet server where the clinician can access it. One example of such a patient management system is the LATITUDE Patient Management System available from Boston Scientific CRM. Many examples of various configurations for patient management systems are described in U.S. Patent Application Publication No. 2006-0106433, titled ADVANCED PATIENT MANAGEMENT SYSTEM INCLUDING INTERROGATOR/TRANSCEIVER UNIT, which is hereby incorporated herein by reference. For example, the processing device 24 located outside of the patient's body and shown in FIG. 6 is or is a part of a patient management system in an embodiment of the invention.

In one embodiment, communication device 32 is configured to transmit wireless signals from turbulence sensor 30 to processing device 24, and processing device 24 is configured to receive wireless signals from communication device 32. In a separate embodiment, an electrical conductor such as a wire is provided that is in electrical communication between sensing unit 22 and processing device 24, to allow the transmission of signals therebetween. In another embodiment, an optical conductor is provided that is in optical communication with the sensing unit 22 and the processing device 24 to allow signal transmission therebetween. In yet another embodiment, communication device 32 is configured to transmit ultrasound signals and processing device 24 is configured to receive ultrasound signals.

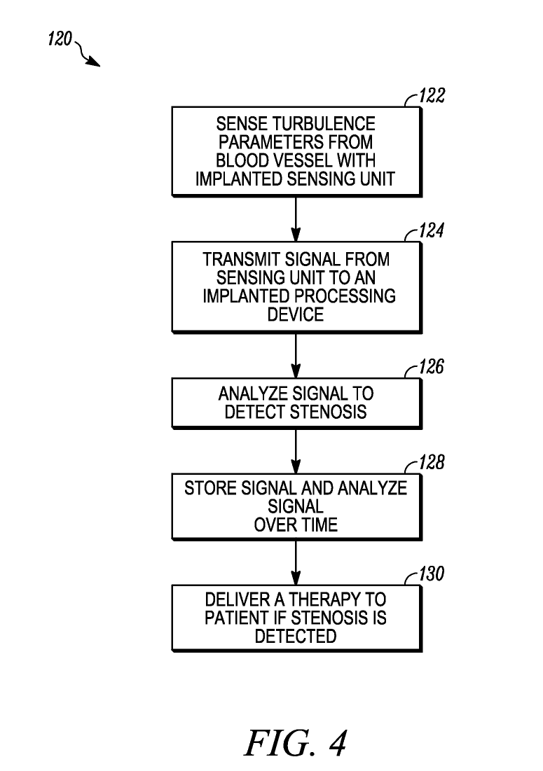

A further embodiment of the invention relates to a method of detecting stenosis in a patient's vasculature. The method 120 is depicted in FIG. 4. Method 120 includes the step 122 of sensing pressure and/or pressure waves within a blood vessel using an implanted sensing unit. Method 120 further includes step 124 of transmitting a signal from the sensing unit to an implanted processing device configured to receive signals from the sensing unit. The processing device then analyzes the signal in step 126 to detect stenosis from the sensing unit signal. In an alternative embodiment, step 124 of transmitting the signal involves transmitting the signal wirelessly. In another embodiment, the method includes additional step 128 of storing the sensing unit signal and analyzing the sensing unit signal over time to detect stenosis from the sensing unit signal. Yet another embodiment includes the additional step 130 of automatically delivering a therapy to the patient by activating an implanted therapy device when stenosis is detected in step 126. Step 130 may involve activating a drug pump. Alternatively, step 130 may involve delivering electrical stimulation to the patient's heart.

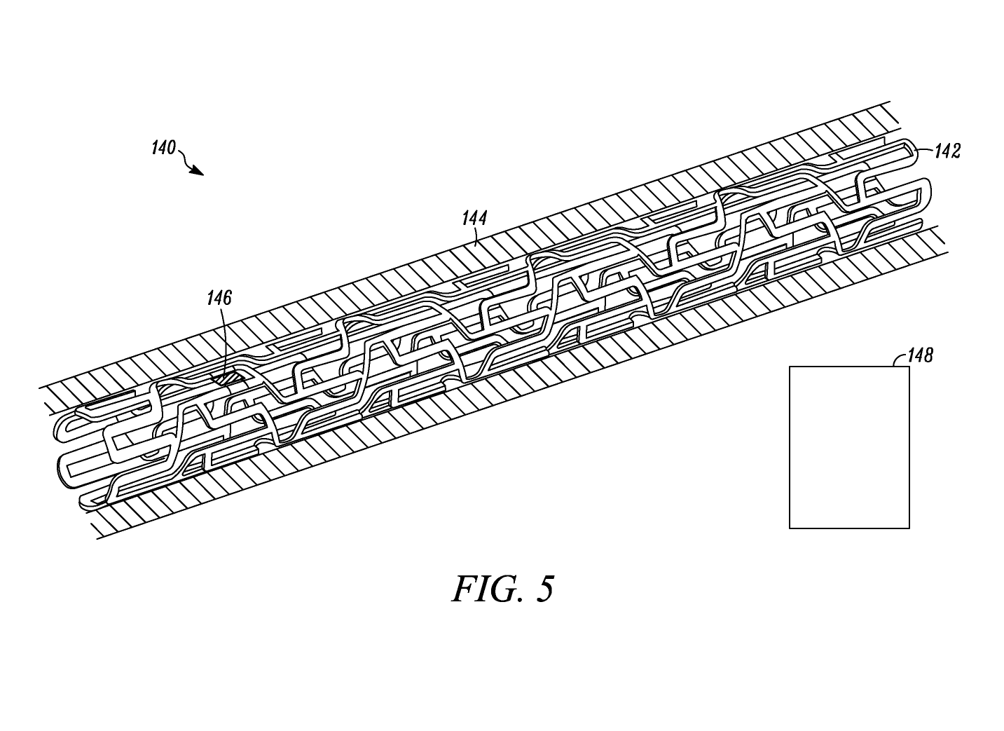

Yet another embodiment of the invention is depicted in FIG. 5. The embodiment of FIG. 5 relates to an intravascular medical device. Specifically, the embodiment of FIG. 5 is a stent system. Stent system 140 includes a stent 142 positioned within a blood vessel 144 of a patient. Stent 142 is an expandable, generally cylindrical structure that is configured to be placed in a body lumen such as a blood vessel and to exert a radial pressure on the body lumen. The construction of stent 142 is according to the general principles known to a person of skill in the art, and as such generally is constructed from a series of interlocking thin metal pieces or wires. Stent 142 is particularly useful for applying radial pressure to a blood vessel to help prevent narrowing of the vessel and to maintain sufficient blood flow. Stent system 140 further includes a turbulence sensor 146 that is attached to stent 142. Turbulence sensor 146 is configured to sense turbulence within vessel 144. For example, turbulence sensor 146 may be an accelerometer that is configured to sense vibration waves within vessel 144. Alternatively, turbulence sensor 146 may be a microphone that is configured to sense acoustic waves within vessel 144. Turbulence sensor 146 may also be a pressure transducer that senses pressure within vessel 144. Yet other embodiments of turbulence sensor 146 are usable. The design of stent 142 may be such that the physical properties, such as mechanical resonance or mechanical filter characteristics, may be used to enhance or “tune” the sensitivity of the turbulence sensor 146 to the turbulence signal of interest. The turbulence sensor 146 may be an integral part of stent 142.

Turbulence sensor 146 is configured to generate a signal that corresponds to a level of turbulence within blood vessel 144, and is further configured to transmit the signal. Turbulence sensor 146 may be attached to stent 142 by any of a number of different means. For example, turbulence sensor 146 may be sutured to stent 142. Alternatively, turbulence sensor 146 may be micro-welded to stent 142, or sensor 146 may be integrally wound into wires that form stent 142. Other forms of attachment may also be used. Turbulence sensor 146 may also include additional structures, such as a power source and a communicating device for transmitting a signal.

Stent system 140 further includes a processing device 148. Processing device 148 is configured to receive a signal generated by turbulence sensor 146. Processing device 148 uses the signal received from the turbulence sensor 146 to detect the presence of stenosis within blood vessel 144. In one embodiment, processing device 148 is located inside the patient's body. In another embodiment, processing device 148 is located outside the patient's body. In one embodiment, processing device 148 is configured to provide an indication of the presence of stenosis. For example, processing device 148 may be configured to send a signal through telemetry to a device that can display a perceptible indication, such as a message on a screen, to a person, such as a physician. In another embodiment, processing device 148 is configured to control a medical therapy in response to a detection of stenosis. For example, in one embodiment, stent 142 includes a drug coating, and processing device 148 is configured to control the rate of release of the drug coating in response to the detection of stenosis.

Certain implanted medical devices, such as cardiac rhythm management devices, include sensors such as accelerometers, microphones and pressure sensors that can be tuned to detect heart murmurs caused by faulty heart valves. A problem arises, however, in diagnosing a specific heart defect based on the existence of a detected heart murmur. Particularly, it can be difficult to determine whether a murmur arises from the right or left side of the heart. It is important to know which side of the heart a murmur originates from for diagnostic and treatment purposes. For example, mitral regurgitation is an indication of disease progression in a heart failure patient and is manifested as a left-sided murmur. Therefore, being able to distinguish between a right-sided murmur and a left-sided murmur can provide additional information about a patient's condition.

It has been found that it is possible to determine which side of the heart the detected murmur originated from by comparing the intensity of the murmur to the subject's respiration cycle. Inspiration causes a decrease in intrathoracic pressure, allowing air to enter the lungs. This decrease in intrathoracic pressure also causes an increase in the venous return to the right side of the heart. Therefore, right sided murmurs generally increase with inspiration. The increased volume of blood entering the right sided chambers of the heart restricts the amount of blood entering the left sided chambers of the heart. This causes left sided murmurs to generally decrease in intensity during inspiration.

During expiration, the opposite hemodynamic changes occur. Expiration causes an increase in intrathoracic pressure, expelling air from the lungs. This increase in intrathoracic pressure causes a decrease in the venous return to the right side of the heart. Therefore, right sided murmurs generally decrease with expiration. The increase in intrathoracic pressure also causes an increase in the amount of blood entering the left sided chambers of the heart. This causes left sided murmurs to generally increase in intensity during expiration.

Based on this relationship between murmur intensity and respiration, right sided and left side murmurs can be accurately distinguished. By way of example, the intensity of a murmur can be monitored over a period of time using an accelerometer or other device. If the intensity of the murmur increases during inspiration and decreases during expiration, then it is most likely a right sided murmur. However, if the intensity of the murmur decreases during inspiration but increases during expiration, then it is most likely a left sided murmur.

FIG. 8 shows one example of a method of differentiating between left-sided and right-sided murmurs. A cardiac signal is obtained from a sensor and then processed. A respiratory signal is also obtained from a sensor and then processed. If a murmur is detected, then the timing of the murmur with respect to inspiration and expiration is evaluated. If the intensity of the murmur increases during inspiration and decreases during expiration, then it is most likely a right sided murmur. However, if the intensity of the murmur decreases during inspiration but increases during expiration, then it is most likely a left sided murmur.

Devices can be configured to differentiate between left sided and right sided murmurs according to the method described herein. Specifically, a device can be configured to receive cardiac and respiratory sensor data, detect a heart murmur, and determine whether the murmur is associated with the left or right side of the heart.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

The above specification provides a complete description of the structure and use of the invention. Since many of the embodiments of the invention can be made without parting from the spirit and scope of the invention, the invention resides in the claims.

What is claimed is:

1. A system for detecting stenosis in a patient, the system comprising:
an implantable sensing unit having a turbulence sensor and a communication device for transmitting a signal from the turbulence sensor;
a cardiac sensor for generating a signal corresponding to cardiac activity; and
a processing device configured to receive signals from the sensing unit and from the cardiac sensor;
the processing device configured to determine a time window corresponding to cardiac activity, to determine a turbulence level from the turbulence signal within the time window, and to detect the presence of stenosis from the turbulence level.

2. The system of claim 1, wherein the turbulence sensor and communication device are within a common housing, and wherein the processing device is located in a second housing that is separate from the common housing of the turbulence sensor and communication device.

3. The system of claim 1, wherein the time window corresponding to cardiac activity is selected from the group consisting of a systolic portion of a cardiac cycle, a diastolic portion of a cardiac cycle, and a complete cardiac cycle.

4. The system of claim 1, wherein the turbulence sensor comprises an acoustic sensor.

5. The system of claim 1, wherein the turbulence sensor comprises a vibration sensor.

6. The system of claim 1, wherein the processing device is implantable.

7. The system of claim 1, wherein the processing device is configured to be positioned outside of a patient's body.

8. The system of claim 1, wherein:
the communication device of the sensing unit is configured to transmit the signal by telemetry to the processing device, and
the processing device is configured to receive the telemetric signal from the communication device.

9. The system of claim 1, further comprising an electrical conductor that is in electrical communication between the sensing unit and the processing device.

10. The system of claim 1, further comprising an optical conductor that is in optical communication between the sensing unit and the processing device.

11. The system of claim 1, wherein the sensing unit is proximate to the patient's coronary artery.

12. The system of claim 1, wherein the sensing unit is proximate to the patient's carotid artery.

13. The system of claim 1, wherein the processing device is located inside of a cardiac rhythm management device.

14. The system of claim 13, wherein the cardiac rhythm management device is selected from the group consisting of a pacemaker, a defibrillator, a cardiac resynchronization device, and a neural stimulation device.

15. The system of claim 1, further comprising a respiratory sensor for generating a signal corresponding to respiratory activity, and wherein the processing device is further configured to receive the signal from the respiratory sensor, to synchronize the respiratory signal to the turbulence signal and the cardiac activity signal, and to detect stenosis from the received signals.

16. The system of claim 1, wherein the cardiac sensor comprises a sensor selected from the group consisting of a microphone, an accelerometer, an impedance sensor, and a cardiac electrical signal monitor.

17. The system of claim 1, wherein the turbulence signal is trended over time to detect complete occlusion of the blood vessel.

18. A method of detecting stenosis that occurs in a patient's vasculature, the method comprising:
sensing turbulence that occurs within a blood vessel using an implanted sensing unit;
sensing the patient's cardiac activity;
transmitting signals representing turbulence and cardiac activity to a processing device; and
analyzing the turbulence and cardiac activity signals within the processing device to determine a time window corresponding to cardiac activity, determining a turbulence level from the turbulence signal within the time window, and detecting the presence of stenosis within a blood vessel.

19. The method of claim 18, wherein the implanted sensing unit is separate from the processing device.

20. The method of claim 18, wherein the implanted sensing unit comprises an acoustic sensor.

21. The method of claim 18, wherein the implanted sensing unit comprises a vibration sensor.

22. The method of claim 18, wherein the implanted sensing unit comprises a pressure sensor.

23. The method of claim 18, wherein the processing device is implantable.

24. The method of claim 18, wherein the processing device is configured to be positioned outside of a patient's body.

25. The method of claim 18, wherein the processing device is a cardiac rhythm management device.

26. The method of claim 18 further comprising the step of transmitting signals representing turbulence and cardiac activity to an external device, wherein the external device comprises one of a group consisting of a programmer, a repeater and a remote patient management system.

27. The method of claim 26, wherein the external device comprises the remote patient management system, wherein the remote patient management system is configured to make turbulence and cardiac activity information accessible to a clinician.

28. A stent system comprising:
an expandable, generally cylindrical structure configured to be placed in a body lumen and to exert radial pressure on the body lumen;
a turbulence sensor attached to the cylindrical structure, the turbulence sensor being configured to transmit a signal; and
a processing device configured to receive the signal from the turbulence senor and to analyze the signal to detect the presence of stenosis within the body lumen.

29. The stent system of claim 28, wherein the turbulence sensor comprises an acoustic sensor.

30. The stent system of claim 28, wherein the turbulence sensor comprises a vibration sensor.

31. The stent system of claim 28, wherein the turbulence sensor comprises a pressure sensor.

32. The stent system of claim 28, wherein the processing device is implantable.

33. The stent system of claim 28, wherein the processing device is outside of the patient's body.

34. The stent system of claim 28, wherein the processing device is a cardiac rhythm management device.
